(19) United States
(12) Patent Application Publication
(10) Pub. No.: US 2025/0282801 A1
(43) Pub. Date: Sep. 11, 2025

(54) TRITYL-BASED ALCOHOL PROTECTING GROUP

(71) Applicant: SynCrest Inc., Kanagawa (JP)

(72) Inventors: Pengyu XU, Kanagawa (JP); Shin YONEYAMA, Kanagawa (JP)

(73) Assignee: SynCrest Inc., Kanagawa (JP)

(21) Appl. No.: 19/105,038

(22) PCT Filed: Aug. 21, 2023
(86) PCT No.: PCT/JP2023/029912

§ 371 Date: Feb. 20, 2025

(30) Foreign Application Priority Data
Aug. 22, 2022 (JP) 2022-131789

(51) Int. Cl. C07F 7/08; C07H 23/00

(52) U.S. Cl. CPC C07F 7/0805; C07H 23/00

(57) ABSTRACT

An object of the present invention is to provide a novel trityl-based alcohol protecting group that is useful in synthesizing nucleic acids. Provided is a trityl-based alcohol protecting group.

### TECHNICAL FIELD

The present invention relates to a trityl-based alcohol protecting group.

### BACKGROUND ART

NPL 1 and NPL 2 disclose a method for synthesizing a nucleic acid using a 4,4′-dimethoxytrityl group (a DMTr protecting group).

### CITATION LIST

### Non-Patent Literature

NPL 1: Tetrahedron Letters, 22, 1859-1862, 1981
NPL 2: J. Am. Chem. Soc., 85, 3821-3827, 1963

### SUMMARY OF INVENTION

### Technical Problem

An object of the present invention is to provide a novel trityl-based alcohol protecting group that is useful in synthesizing nucleic acids.

### Solution to Problem

The present inventors have conducted extensive research to achieve the above object. As a result, the present inventors found that by introducing at least one triple bond into a protecting group, such as a 4,4′-dimethoxytrityl group (a DMTr protecting group), a novel trityl-based alcohol protecting group that is useful in synthesizing nucleic acids can be produced.

The present inventors conducted further research based on this finding and have accomplished the present invention.

Specifically, the present invention includes the following trityl-based alcohol protecting groups.

### Item 1

A compound represented by the following formula (1):

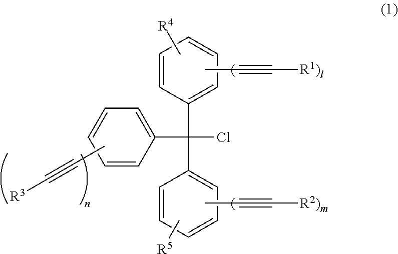

(wherein
R¹, R², and R³ are same or different, and each represents a hydrogen atom, an alkyl group, a trimethylsilyl group (TMS, —Si(CH₃)₃), an acetyl group (Ac), a benzoyl group (Bz), an ester group, an alkyl ester group, an aryl group, an alkyl alcohol group, a nitrile group (CN), a group represented by

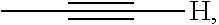

or a group represented by

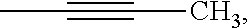

wherein l, m, and n each represent an integer of 0, 1, or 2 (provided that l, m, and n are not 0 at the same time), and
R⁴ and R⁵ are the same or different, and each represents an electron-donating group).

As a trityl-based alcohol protecting group, the compound represented by formula (1) of the present invention is excellent in controlling the ease of deprotection. The compound represented by formula (1) of the present invention enables improvement of a substituent, such as a DMTr protecting group, and adjustment of the deprotection conditions to thereby increase the yield of the nucleic acid synthesized. Based on the triple bond introduced into the trityl-based alcohol protecting group, the compound represented by formula (1) of the present invention can change cationic properties of the trityl moiety and allows the protecting group to be readily removed for deprotection.

The triple bond introduced into the compound represented by formula (1) of the present invention in the form of a trityl-based alcohol protecting group can be detected by Raman spectroscopy. Based on the trityl-based alcohol protecting group, the compound represented by formula (1) of the present invention allows for detection of quantifiable peaks in the silent region and enables quantification of the protecting group without being disturbed by other impurities and is excellent in tracking various reactions.

### Advantageous Effects of Invention

The present invention can newly provide a trityl-based alcohol protecting group that is useful in synthesizing nucleic acids.

### DESCRIPTION OF EMBODIMENT

The present invention is described in detail below.

In the present specification, the terms “comprise” and “contain” encompass the concepts of comprising, consisting essentially of, and consisting of.

In the present specification, the numerical range indicated by “A to B” means A or more and B or less, unless otherwise specified.

### [1] Trityl-Based Alcohol Protecting Group

The trityl-based alcohol protecting group of the present invention is a compound represented by the following formula (1):

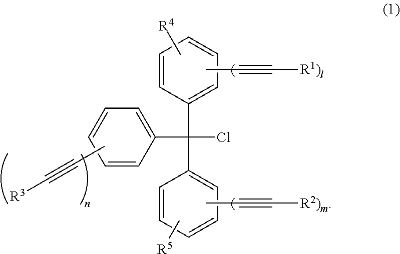

In formula (1), R¹, R², and R³ are same or different, and each represents a hydrogen atom, an alkyl group, a trimethylsilyl group, an acetyl group, a benzoyl group, an ester group, an alkyl ester group, an aryl group, an alkyl alcohol group, a nitrile group, a group represented by

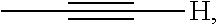

or a group represented by formula (6)

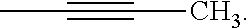

In formula (1), l, m, and n each represent an integer of 0, 1, or 2, provided that l, m, and n are not 0 at the same time.

In formula (1), R⁴ and R⁵ may be same or different, and each represents an electron-donating group.

R¹, R², and R³

In formula (1), R¹, R², and R³ are same or different, and each represents a hydrogen atom (H), an alkyl group, a trimethylsilyl group (TMS, —Si(CH₃)₃), an acetyl group (Ac), a benzoyl group (Bz), an ester group, an alkyl ester group, an aryl group, an alkyl alcohol group, a nitrile group (CN), a group represented by

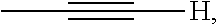

or a group represented by

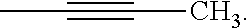

The alkyl group is preferably, for example, a methyl group (—CH₃, Me), an ethyl group (Et), an n-butyl group (n-Bu), or a t-butyl group (t-Bu).

The ester group is preferably, for example, a methyl ester group (COOMe) or an ethyl ester group (COOEt).

The alkyl ester group is preferably, for example, a 3-carboxypropyl group ((CH₂)₃COOH) or a 3-methyloxycarbonylpropyl group ((CH₂)₃COOMe).

The aryl group is preferably, for example, a phenyl group (Ph), a 4-methyloxycarbonylbenzene group (4-COOMe-C₆H₄), a 4-formylbenzene group (4-CHO—C₆H₄), a 4-methylhydroxybenzene group (4-CH₂OH—C₄H₆), or a 3,5-dimethoxybenzene group (3,5-OMe-C₆H₄).

The alkyl alcohol group (e.g., CH₂OH, (CH₂)₂OH, (CH₂)₃OH), CH₂OCO₂(4-NO₂-C₄H₆), or (CH₂)₃OCO₂(4-NO₂-C₄H₆) is preferably, for example, a methyl hydroxyl group (CH₂OH), an ethyl hydroxyl group ((CH₂)₂OH), a propyl hydroxyl group ((CH₂)₃OH), a methyl(4-nitrobenzo)oxycarbonyl group (CH₂OCO₂(4-NO₂-C₄H₆)), or a propyl(4-nitrobenzo)oxycarbonyl group ((CH₂)₃OCO₂(4-NO₂-C₄H₆)).

In formula (1), the substituent of a triple bond (alkyne) including R¹:

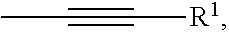

the substituent of a triple bond (alkyne) including R²:

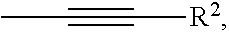

and
the substituent of a triple bond (alkyne) including R³:

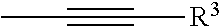

may be bonded at any of the ortho-, meta-, or para-positions.

In formula (1), the substituent of a triple bond including R¹, the substituent of a triple bond including R², and the substituent of a triple bond including R³ are each bonded, for example, at a para-position with respect to the carbon atom of the benzene ring bonded to the central carbon atom (the “—C” in “—C(C₆H₅)₃”) of the trityl group (—C(C₆H₅)₃); and
l, m, and n each represent an integer of 0, 1, or 2,
provided that l, m, and n are not 0 at the same time,
l is the number of substituents in the triple bond including R¹ (alkyne),
m is the number of substituents in the triple bond including R² (alkyne), and
n is the number of substituents in the triple bond including R³ (alkyne).

In formula (1), at least one of R¹, R², and R³ is preferably a trimethylsilyl group.

R⁴ and R⁵

In formula (1), R⁴ and R⁵ may be same or different, and each represents an electron-donating group.

The electron-donating group is preferably
a linear or branched alkyl group, such as methyl, ethyl, n-propyl, isopropyl, n-butyl, isobutyl, or tert-butyl;
a cycloalkyl group, such as cyclopropyl, cyclobutyl, or cyclopentyl;
an alkenyl group, such as vinyl, allyl, or 1-propenyl;
a cycloalkenyl group, such as 1-cyclopentenyl or 1-cyclohexenyl;
an aryl group, such as phenyl;
an aralkyl group, such as benzyl or phenethyl;
an alkoxy group, such as methoxy, ethoxy, n-propoxy, iso-propoxy, n-butoxy, or tert-butoxy;
an aryloxy group, such as phenyloxy;
a hydroxy group;
an amino group;
an amino group having one or two alkyl groups, such as methylamino, ethylamino, n-propylamino, dimethylamino, or diethylamino;
an amino group having one or two aryl groups, such as phenylamino; or a hydroxyamino group.

The electron-donating group is preferably an alkyl sulfide group, such as a methyl sulfide group (methylthio group), an ethyl sulfide group, an n-propyl sulfide group, an isopropyl sulfide group, an n-butyl sulfide group, an isobutyl sulfide group, or a tert-butyl sulfide group.

The electron-donating group is more preferably, for example, a linear or branched alkyl group, an alkoxy group, a hydroxy group, an amino group, or an amino group having one or two alkyl group, even more preferably an alkoxy group, a hydroxy group, or an amino group, and particularly preferably an alkoxy group having 1 to 4 carbon atoms.

The electron-donating group is preferably an alkoxy group, such as methoxy (—OMe), ethoxy (—OEt), n-propoxy (—OnPr), isopropoxy (—OiPr), n-butoxy (—OnBu), or tert-butoxy (—OtBu).

The electron-donating group is particularly preferably a linear or branched alkyl group, such as methyl, ethyl, n-propyl, isopropyl, n-butyl, isobutyl, or tert-butyl.

The electron-donating group is particularly preferably an alkylsulfide group, such as a methylsulfide group (e.g., a methylthio group, —S-Me, —S—CH₃) or an ethylsulfide group (—S-Et).

In formula (1), R⁴ and R⁵ may be each bonded at any of the ortho-, meta-, or para-positions.

In formula (1), R⁴ and R⁵ are each preferably, for example, bonded at a para-position with respect to the carbon atom of a benzene ring bonded to the central carbon atom (“—C” in “—C(C₆H₅)₃”) of the trityl group (—C(C₆H₅)₃).

The trityl-based alcohol protecting group of the present invention is a protecting group, such as a 4,4′-dimethoxytrityl group (a DMTr protecting group), that has at least one triple bond introduced into the protecting group. The triple bond introduced into the trityl-based alcohol protecting group conjugates with a DMTr protecting group or like protecting group to thereby change cationic properties of the trityl moiety of the DMTr protecting group. Due to this feature, the trityl-based alcohol protecting group of the present invention can control the ease of removal of the protecting group from a target nucleic acid.

The DMTr protecting group or like protecting group of the trityl-based alcohol protecting group changes in stability of the cation. For example, under weak acidic conditions, the protecting group forms a trityl-based alcohol protecting group that can be easily or instantaneously removed.

The triple bonds introduced into the trityl-based alcohol protecting group of the present invention produce quantifiable peaks in the silent region in Raman spectroscopy. Due to this feature, the trityl-based alcohol protecting group of the present invention allows for quantitative determination of the protecting group without being interfered with by other impurities.

The trityl-based alcohol protecting group of the present invention is useful in producing nucleic acids.

Embodiments of the present invention are described above; however, the present invention is not limited to these examples. Needless to say, the present invention can be implemented in various forms within the scope not departing from the gist of the present invention.

### EXAMPLES

Embodiments of the present invention are described in more detail below based on Examples.

The present invention is not limited to these Examples.

### EXAMPLES

### [1] Production 1 of Trityl-Based Alcohol Protecting Group (Scheme 1)

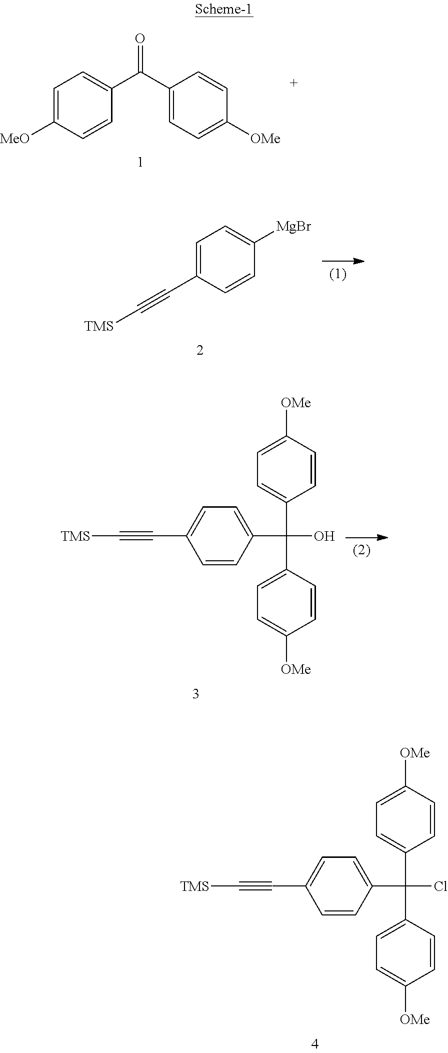

### (1-1) Production of Compound 3 in Scheme 1

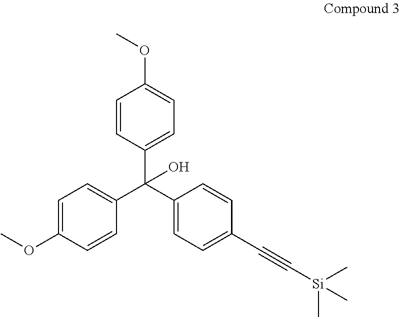

Under an argon atmosphere, magnesium powder (720 mg, 29.6 mmol) and iodine (2 items) were placed in a 100 mL round-bottom flask, and 23 mL of tetrahydrofuran (THF) was added. The resulting mixture was stirred at room temperature. A solution (8.0 mL) of 4-(bromophenylethynyl)trimethylsilane (a precursor of Compound 2 in Scheme 1) (5.00 g, 19.7 mmol) in THE was added dropwise thereto and the resulting mixture was stirred at room temperature for 2 hours to prepare a Grignard reagent of 4-(bromophenylethynyl)trimethylsilane (Compound 2 in Scheme 1).

4,4′-Dimethoxybenzophenone (Compound 1 in Scheme 1) (4.29 g, 17.7 mmol) was added to this reaction liquid, and the resulting mixture was stirred at room temperature for 3 days. After completion of the reaction was confirmed, an aqueous ammonium chloride solution was added to the reaction liquid, and the resulting mixture was extracted with ethyl acetate. The organic layer was dried over magnesium sulfide and concentrated.

The residue was purified by silica gel column chromatography (hexane/ethyl acetate=19/1→9/1) to obtain the target compound (Compound 3 in Scheme 1) (4.63 g, 11.1 mmol) as a white solid.

¹H-NMR (CDCl₃): δ 7.42-7.38 (m, 2H), 7.25-7.21 (m, 2H), 7.16-7.11 (m, 4H), 6.85-6.80 (m, 4H), 3.80 (s, 6H), 2.66 (s, 1H), 0.24 (s, 9H)

¹³C-NMR (CDCl₃): δ 158.8, 147.6, 139.0, 131.5, 129.1, 127.6, 121.8, 113.3, 104.9, 94.3, 81.3, 55.3, 0.0

### (1-2) Production of Compound 4 (Trityl-Based Alcohol Protecting Group) in Scheme 1

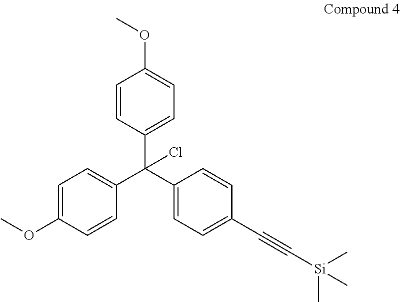

Thionyl chloride (0.17 mL, 2.4 mmol) and N, N-dimethylformamide (DMF) (1 drop) were added to 4-trimethylsilylethynyl-4′,4″-dimethoxyltrityl alcohol (Compound 3 in Scheme 1) (0.20 g, 0.48 mmol), and the resulting mixture was stirred at 45° C. for 1.5 hours.

The stirred mixture was then concentrated and the residue of the target compound was obtained as a red solid (Compound 4 (trityl-based alcohol protecting group) in Scheme 1).

¹H-NMR (CDCl₃): δ 7.41-7.37 (m, 2H), 7.22-7.18 (m, 2H), 7.15-7.10 (m, 4H), 6.84-6.79 (m, 4H), 3.82 (s, 6H), 0.25 (s, 9H)

¹³C-NMR (CDCl₃): δ 159.3, 146.1, 137.4, 131.4, 131.1, 129.7, 122.7, 113.2, 113.1, 104.7, 95.3, 82.4, 55.5, 0.1

### (1-3) Protection of Nucleic Acid by Trityl-Based Alcohol Protecting Group (Scheme 2)

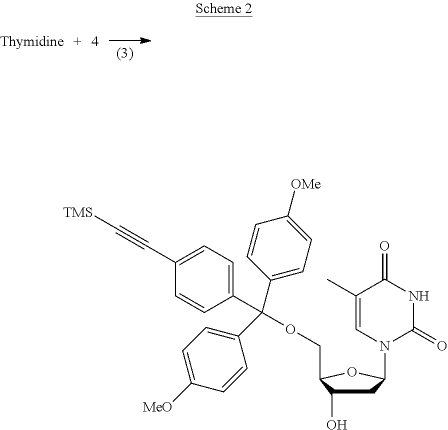

Nucleic Acid Protected with a Trityl-Based Alcohol-Protecting Group

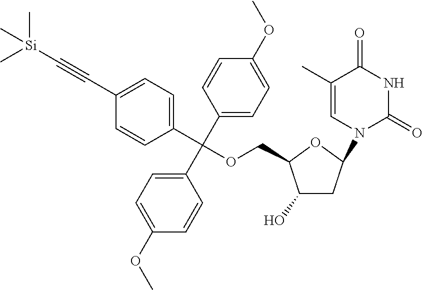 [SMILES: COc1ccc(C(OC[C@H]2O[C@@H](n3cc(C)c(=O)[nH]c3=O)C[C@@H]2O)(c2ccc(C#C[Si](C)(C)C)cc2)c2ccc(OC)cc2)cc1]

Pyridine (2.0 mL) was added to thymidine (nucleic acid) (90 mg, 0.37 mmol) and 4-trimethylsilylethynyl-4′,4″-dimethoxyltrityl chloride (Compound 4 and a trityl-based alcohol protecting group in Schemes 1 and 2) (178 mg, 0.41 mmol), and the resulting mixture was stirred at 50° C. overnight. The stirred mixture was then concentrated, and an aqueous ammonium chloride solution was added. The resulting mixture was extracted with ethyl acetate, and the organic layer was dried over magnesium sulfide and concentrated.

The residue was purified by silica gel column chromatography (hexane/ethyl acetate=1/1→0/1) to obtain the target compound (90 mg, 0.14 mmol) as a white solid (a nucleic acid protected with a trityl-based alcohol protecting group).

¹H-NMR (DMSO-d₆): δ 11.33 (s, 1H), 7.45 (d, 1H, J=1.2 Hz), 7.39-7.37 (m, 4H), 7.25-7.22 (m, 4H), 6.91-6.88 (m, 4H), 6.20 (t, 1H, J=6.6 Hz), 5.30 (d, 1H, J=4.5 Hz), 4.28-4.22 (m, 1H), 3.89-3.85 (m, 1H), 3.74 (s, 6H), 3.23-3.12 (m, 2H), 2.27-2.13 (m, 2H), 1.50 (d, 3H, J=1.2 Hz), 0.21 (s, 9H)

¹³C-NMR (DMSO-d₆): δ 163.7, 158.3, 150.4, 145.8, 135.7, 134.9, 134.5, 131.2, 129.8, 129.7, 127.9, 120.6, 113.3 109.6, 104.9, 94.3, 85.6, 85.3, 83.8, 70.5, 64.0, 55.1, 39.3, 11.8, −0.1

### [2] Production 2 of Trityl-Based Alcohol Protecting Group (Scheme 3)

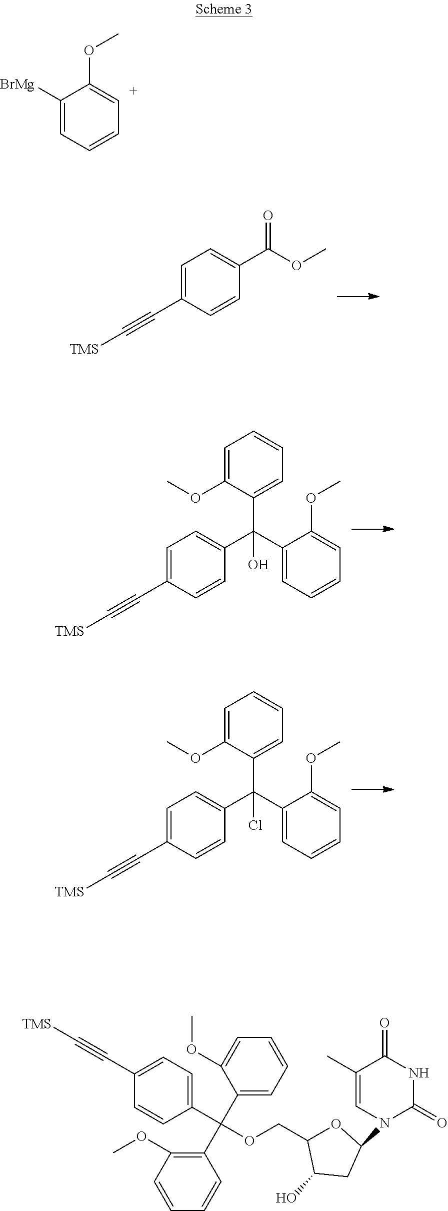

### (2-1) Production of 4-trimethylsilylethynyl-2′,2″-dimethoxyltrityl Alcohol

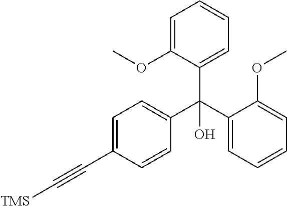 [SMILES: COc1ccccc1C(O)(c1ccc(C#C[Si](C)(C)C)cc1)c1ccccc1OC]

Under argon atmosphere, magnesium powder (306 mg, 12.6 mmol) and iodine (1 item) were placed in a 100 mL eggplant flask, and THF (10 mL) was added. The resulting mixture was stirred at room temperature. A solution (3.5 mL) of 2-bromoanisole (1.57 g, 8.40 mmol) in THF was added dropwise thereto, and the resulting mixture was stirred at a temperature of 40° C. to 50° C. for 1 hour.

Methyl 4-(trimethylsilylethynyl)benzoate (888 mg, 3.82 mmol) was added to this reaction liquid, and the resulting mixture was stirred at room temperature for 1 hour. After completion of the reaction was confirmed, 1N hydrochloric acid was added to the reaction liquid, and the resulting mixture was extracted with ethyl acetate. The organic layer was dried over magnesium sulfide and concentrated.

The residue was purified by silica gel column chromatography (hexane/ethyl acetate=19/1→4/1) to obtain the target compound (1.45 g, 3.48 mmol) as a yellow solid.

¹H-NMR (CDCl₃): δ 7.38-7.34 (m, 2H), 7.32-7.26 (m, 2H), 7.22-7.18 (m, 2H), 7.00-6.97 (m, 2H), 6.93-6.85 (m, 4H), 5.26 (s, 1H), 3.52 (s, 6H), 0.24 (s, 9H)

¹³C-NMR (CDCl₃): δ 157.5, 147.7, 133.6, 131.1, 129.5, 129.0, 127.8, 121.1, 120.6, 112.4, 105.7, 93.7, 80.9, 55.6, 0.2

### (2-2) Production of 4-trimethylsilylethynyl-2′,2″-dimethoxyltrityl Chloride

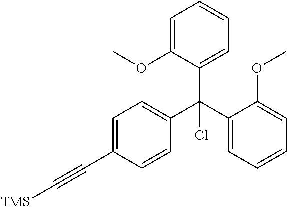 [SMILES: COc1ccccc1C(Cl)(c1ccc(C#C[Si](C)(C)C)cc1)c1ccccc1OC]

Ethyl acetate (2.4 mL) and thionyl chloride (0.35 mL, 4.8 mmol) were added to 4-trimethylsilylethynyl-2′,2″-dimethoxyltrityl alcohol (0.20 g, 0.48 mmol), and the resulting mixture was stirred at 45° C. for 4 hours. The stirred mixture was concentrated to obtain a residue of the target compound as a beige solid.

¹H-NMR (CDCl₃): δ 7.37-7.27 (m, 6H), 7.15-7.11 (m, 2H), 6.92-6.84 (m, 4H), 3.51 (s, 6H), 0.25 (s, 9H) ¹³C-NMR (CDCl₃): δ 157.7, 145.0, 132.0, 130.7, 130.1, 129.8, 129.2, 121.5, 120.1, 112.8, 105.3, 94.3, 80.9, 55.6, 0.1

### (2-3) Protection of Nucleic Acid by Trityl-Based Alcohol Protecting Group

### 5′-O-(4-Trimethylsilylethynyl-2′,2″-dimethoxyltrityl)-thymidine

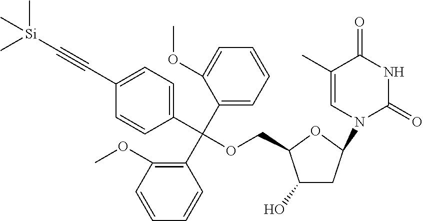 [SMILES: COc1ccccc1C(OC[C@H]1O[C@@H](n2cc(C)c(=O)[nH]c2=O)C[C@@H]1O)(c1ccc(C#C[Si](C)(C)C)cc1)c1ccccc1OC]

Pyridine (1.0 mL) was added to thymidine (67 mg, 0.28 mmol) and 4-trimethylsilylethynyl-2′,2″-dimethoxyltrityl chloride (190 mg, 0.44 mmol), and the resulting mixture was stirred at 50° C. for 3 hours and then concentrated. After adding water, the resulting mixture was extracted with ethyl acetate. The organic layer was dried over magnesium sulfide and concentrated.

The residue was purified by silica gel column chromatography (hexane/ethyl acetate 1/1→0/1) to obtain the target compound (21 mg, 0.033 mmol) as a white solid.

¹H-NMR (DMSO-d₆): δ 11.33 (s, 1H), 7.49 (d, 1H, J=0.9 Hz), 7.43-7.39 (m, 1H), 7.35-7.24 (m, 7H), 7.01-6.90 (m, 4H), 6.20 (t, 1H, J=6.6 Hz), 5.31 (d, 1H, J=4.5 Hz), 4.38-4.33 (m, 1H), 3.91-3.88 (m, 1H), 3.40 (s, 3H), 3.38 (s, 3H)), 3.22 (dd, 1H, J=10.5, 4.5 Hz), 3.13 (dd, 1H, J=10.5, 2.7 Hz), 2.25-2.08 (m, 2H), 1.42 (d, 3H, J=0.9 Hz), 0.19 (s, 9H)

¹³C-NMR (DMSO-d₆): δ 163.7, 157.2, 156.9, 150.4, 145.8, 135.8, 130.0, 129.7, 129.3, 129.3, 129.1, 128.2, 128.0, 120.2, 120.0, 119.5, 112.3, 112.2, 109.5, 105.5, 93.4, 85.5, 84.4, 83.8, 70.5, 64.5, 54.9, 54.8, 39.6, 11.8, −0.1

### [3] Production 3 of Trityl-Based Alcohol Protecting Group (Scheme 4)

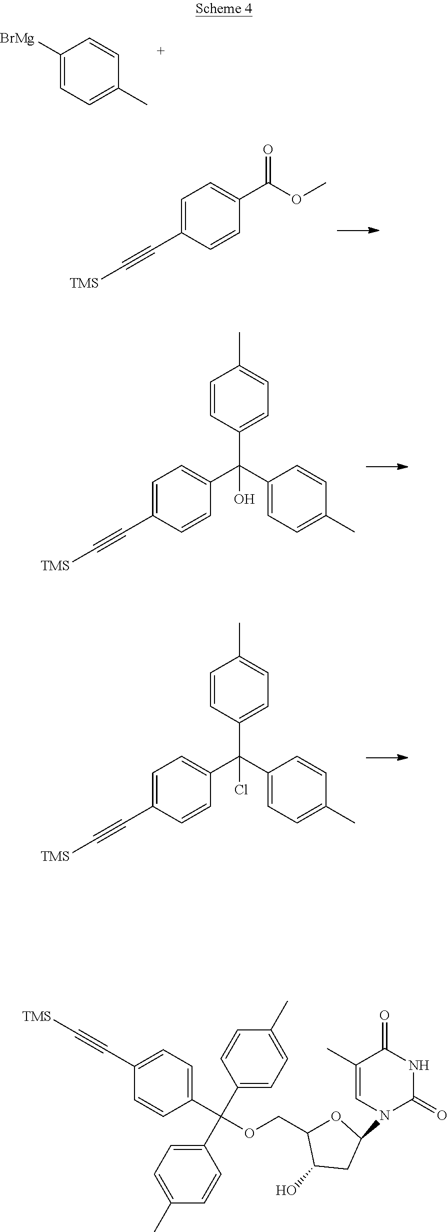

### (3-1) Production of 4-trimethylsilylethynyl-4′,4″-dimethyltrityl Alcohol

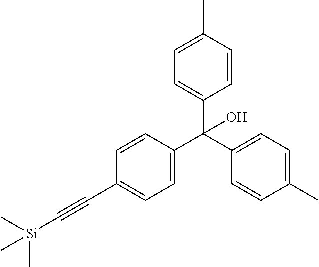

Under argon atmosphere, magnesium powder (237 mg, 9.77 mmol) and iodine (2 items) were placed in a 100 mL eggplant flask, and THF (10 mL) was added. The resulting mixture was stirred at room temperature. A solution (3.5 mL) of 4-bromotoluene (1.11 g, 6.51 mmol) in THE was added dropwise thereto, and the resulting mixture was stirred at 50° C. for 1 hour.

Methyl 4-(trimethylsilylethynyl)benzoate (688 mg, 2.96 mmol) was added to this reaction liquid, and the resulting mixture was stirred at room temperature for 2.5 minutes. 1N HCl was then added to the reaction liquid, and the resulting mixture was extracted with ethyl acetate. The organic layer was dried over magnesium sulfide and concentrated.

The residue was purified by silica gel column chromatography (hexane/ethyl acetate=98/2→95/5) to obtain the target compound (750 mg, 1.95 mmol) as a yellow solid.

¹H-NMR (CDCl₃): δ 7.42-7.38 (m, 2H), 7.26-7.22 (m, 2H), 7.11 (s, 8H), 2.69 (s, 1H), 2.34 (s, 6H), 0.24 (s, 9H)

¹³C-NMR (CDCl₃): δ 147.6, 143.9, 137.2, 131.6, 128.8, 127.9, 127.9, 122.0, 105.1, 94.5, 81.8, 21.2, 0.1

### (3-2) Production of 4-trimethylsilylethynyl-4′,4″-dimethyltrityl Chloride

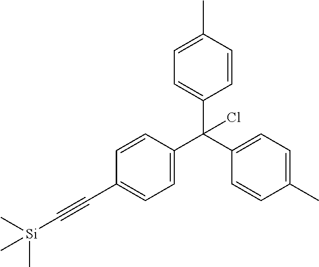

Thionyl chloride (0.16 mL, 2.2 mmol) was added to 4-trimethylsilylethynyl-4′,4″-dimethyltrityl alcohol (170 mg, 0.44 mmol), and the resulting mixture was stirred at 45° C. for 1.5 hours. The stirred mixture was then concentrated to give the target compound (177 mg, 0.44 mmol) as a white-yellow solid.

¹H-NMR (CDCl₃): δ 7.40-7.36 (m, 2H), 7.21-7.17 (m, 2H), 7.09 (s, 8H), 2.36 (s, 6H), 0.25 (s, 9H)

¹³C-NMR (CDCl₃): δ 146.0, 142.3, 137.9, 131.4, 129.7, 129.7, 128.6, 122.6, 104.7, 95.2, 81.3, 21.2, 0.1

### (3-3) Protection of Nucleic Acid Using a Trityl-Based Alcohol Protecting Group

### 5′-O-(4-Trimethylsilylethynyl-4′,4″-dimethyltrityl)-thymidine

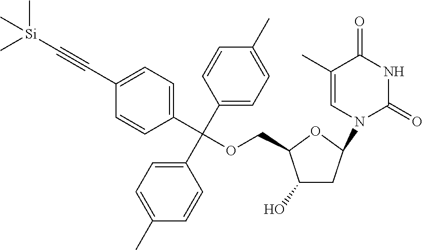 [SMILES: Cc1ccc(C(OC[C@H]2O[C@@H](n3cc(C)c(=O)[nH]c3=O)C[C@@H]2O)(c2ccc(C)cc2)c2ccc(C#C[Si](C)(C)C)cc2)cc1]

4-Trimethylsilylethynyl-4′,4″-dimethyltrityl chloride (165 mg, 0.41 mmol) and thymidine (66 mg, 0.27 mmol) were dissolved in pyridine (1.0 mL), and the resulting mixture was stirred at 50° C. overnight and then concentrated. The organic layer was extracted with ethyl acetate and dried over magnesium sulfide sulfate, and the solvent was distilled off.

The residue was purified by column chromatography (hexane/ethyl acetate=1/1→0/1) to obtain the target compound (87.9 mg, 0.144 mmol) as a white solid.

¹H-NMR (DMSO-d₆): δ 11.33 (s, 1H), 7.45 (d, 1H, J=1.2 Hz), 7.41-7.35 (m, 4H), 7.23-7.20 (m, 4H), 7.15-7.12 (m, 4H), 6.20 (t, 1H, J=6.6 Hz), 5.30 (d, 1H, J=4.5 Hz), 4.27-4.21 (m, 1H), 3.89-3.85 (m, 1H), 3.23-3.11 (m, 1H), 2.27 (s, 6H), 2.24-2.06 (m, 2H), 1.52 (d, 3H, J=1.2 Hz), 0.21 (s, 9H)

¹³C-NMR (DMSO-d₆): δ 163.7, 150.4, 145.3, 140.3, 139.8, 136.5, 135.8, 131.2, 128.6, 128.4, 128.3, 128.2, 120.7, 109.6, 104.9, 94.4, 85.9, 85.3, 83.7, 70.4, 64.2, 39.5, 20.8, 11.8, −0.1

### [4] Production 4 of Trityl-Based Alcohol Protecting Group (Scheme 5)

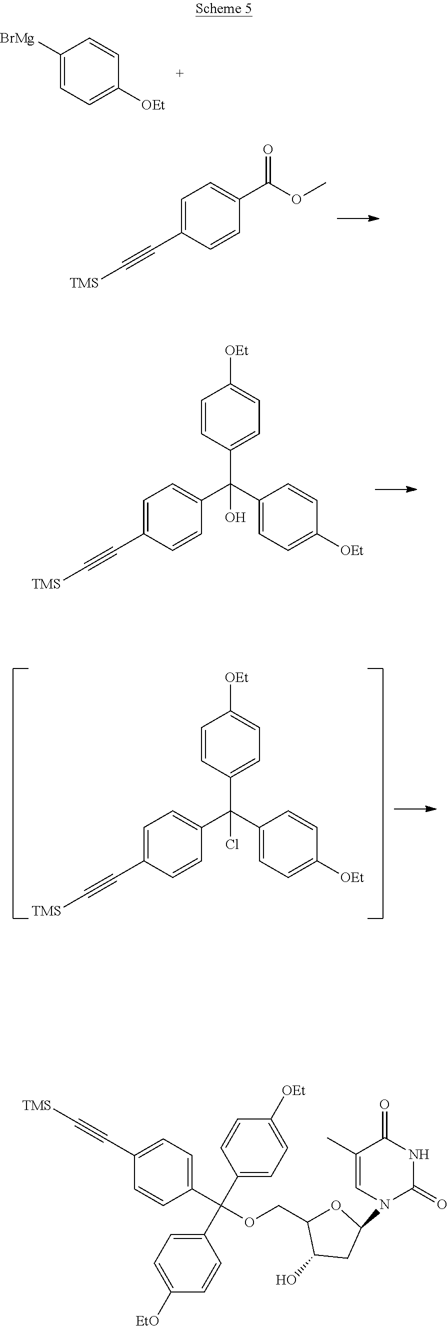

### (4-1) Production of 4-trimethylsilylethynyl-4′,4″-dimethoxytrityl Alcohol

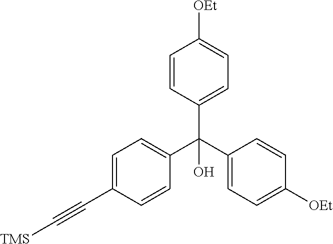 [SMILES: CCOc1ccc(C(O)(c2ccc(C#C[Si](C)(C)C)cc2)c2ccc(OCC)cc2)cc1]

Under argon atmosphere, magnesium powder (237 mg, 9.77 mmol) and iodine (1 item) were placed in a 100 mL eggplant flask, and THF (10 mL) was added. The resulting mixture was stirred at room temperature. A solution (3.5 mL) of 4-bromophenetole (0.928 mL, 6.51 mmol) in THE was added dropwise thereto, and the resulting mixture was stirred at 50° C. or lower for 1 hour.

Methyl 4-(trimethylsilylethynyl)benzoate (570 mg, 2.45 mmol) was added to this reaction liquid, and the resulting mixture was stirred at room temperature for 2.5 hours. 1N HCl was then added to the reaction liquid, and the resulting mixture was extracted with ethyl acetate. The organic layer was dried over magnesium sulfide and concentrated.

The residue was purified by silica gel column chromatography (hexane/ethyl acetate=98/2→95/5) to obtain the target compound (653 mg, 1.47 mmol) as a yellow oil.

¹H-NMR (CDCl₃): δ 7.42-7.38 (m, 2H), 7.26-7.21 (m, 2H), 7.14-7.09 (m, 4H), 6.83-6.78 (m, 4H), 4.02 (q, 4H, J=6.9 Hz), 2.70 (s, 1H), 1.41 (t, 6H, J=6.9 Hz), 0.24 (s, 9H) ¹³C-NMR (CDCl₃): δ 158.3, 147.8, 139.0, 131.6, 129.2, 127.8, 121.9, 113.9, 105.1, 94.4, 81.5, 63.6, 15.0, 0.1

### (4-2) Protection of Nucleic Acid Using a Trityl-Based Alcohol Protecting Group

### 5′-O-(4-Trimethylsilylethynyl-4′,4″-diethoxyltrityl)-thymidine

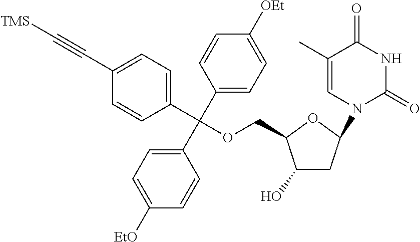 [SMILES: CCOc1ccc(C(OC[C@H]2O[C@@H](n3cc(C)c(=O)[nH]c3=O)C[C@@H]2O)(c2ccc(C#C[Si](C)(C)C)cc2)c2ccc(OCC)cc2)cc1]

4-Trimethylsilylethynyl-4′,4″-diethoxyltrityl alcohol (0.27 g, 0.60 mmol) was dissolved in ethyl acetate (3.0 mL), and thionyl chloride (0.22 mL, 3.0 mmol) was added. The resulting mixture was stirred at 45° C. for 1.5 hours. The stirred mixture was then concentrated to give a product. Subsequently, this product was dissolved in pyridine (3.0 mL), and thymidine (73 mg, 0.30 mmol) was added. The resulting mixture was stirred at 45° C. overnight.

The stirred mixture was concentrated, and the residue was purified by silica gel column chromatography (hexane/ethyl acetate=1/1→0/1) to obtain the target compound (45 mg, 0.067 mmol) as a white solid.

¹H-NMR (DMSO-d₆): δ 11.32 (s, 1H), 7.45 (d, 1H, J=0.9 Hz), 7.39-7.37 (m, 4H), 7.23-7.19 (m, 4H), 6.89-6.86 (m, 4H), 6.20 (t, 1H, J=6.6 Hz), 5.30 (d, 1H, J=4.5 Hz), 4.28-4.22 (m, 1H), 4.00 (q, 4H, J=6.9 Hz), 3.89-3.85 (m, 1H), 3.22-3.12 (m, 2H), 2.27-2.08 (m, 2H), 1.50 (d, 3H, J=0.9 Hz), 1.30 (t, 6H, J=6.9 Hz), 0.21 (s, 9H)

¹³C-NMR (DMSO-d₆): δ 163.6, 157.6, 150.4, 145.8, 135.7, 134.8, 134.4, 131.2, 129.8, 129.7, 127.9, 120.6, 113.7, 109.5, 104.9, 94.3, 85.6, 85.3, 83.7, 70.4, 63.0, 39.4, 14.6, 11.8, −0.1

### [5] Production 5 of Trityl-Based Alcohol Protecting Group (Scheme 6)

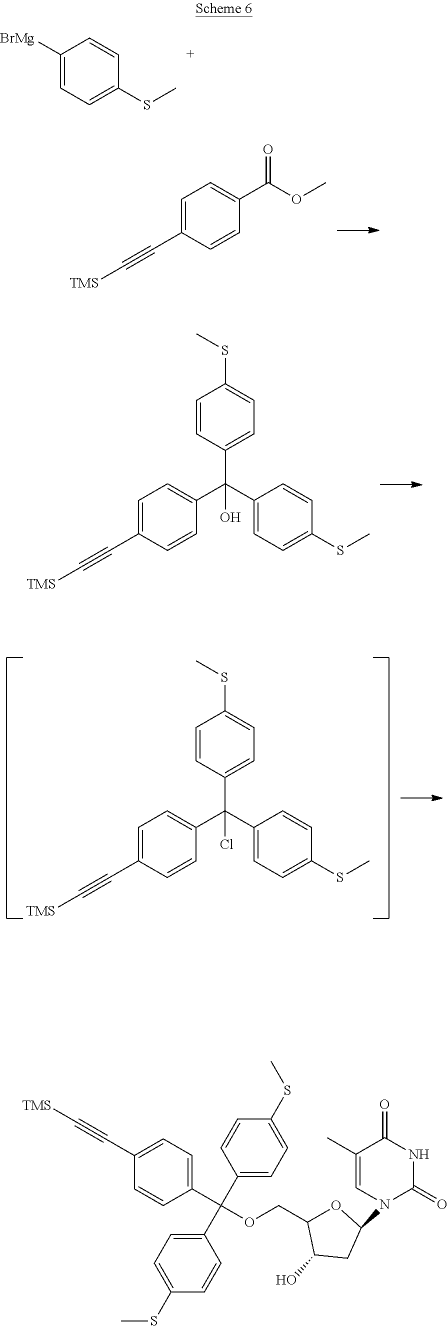

### (5-1) Production of 4-trimethylsilylethynyl-4′,4″-dimethylthiotrityl Alcohol

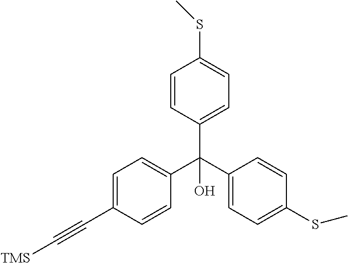 [SMILES: CSc1ccc(C(O)(c2ccc(C#C[Si](C)(C)C)cc2)c2ccc(SC)cc2)cc1]

Under argon atmosphere, magnesium powder (237 mg, 9.77 mmol) and iodine (1 piece) were placed in a 100 mL eggplant flask, and THF (10 mL) was added. The resulting mixture was stirred at room temperature. A solution (3.5 mL) of 4-bromothioanisole (1.32 g, 6.51 mmol) in THF was added dropwise thereto, and the resulting mixture was stirred at 50° C. or lower for 1 hour.

Methyl 4-(trimethylsilylethynyl)benzoate (690 mg, 2.96 mmol) was added to this reaction liquid, and the resulting mixture was stirred at room temperature overnight. 1N HCl was then added to the reaction liquid, the resulting mixture was extracted with ethyl acetate. The organic layer was dried over magnesium sulfide and concentrated.

The residue was purified by silica gel column chromatography (hexane/ethyl acetate=98/2→95/5) to obtain the target compound (1.03 g, 2.29 mmol).

¹H-NMR (CDCl₃): δ 7.43-7.38 (m, 2H), 7.24-7.12 (m, 10H), 2.69 (s, 1H), 2.47 (s, 6H), 0.24 (s, 9H)

¹³C-NMR (CDCl₃): δ 147.0, 143.3, 138.0, 131.7, 128.4, 127.8, 126.1, 122.3, 104.9, 94.8, 81.5, 15.8, 0.1

### (5-2) Protection of Nucleic Acid Using a Trityl-Based Alcohol Protecting Group

### 5′-O-(4-Trimethylsilylethynyl-4′,4″-dimethylthiotrityl)-thymidine

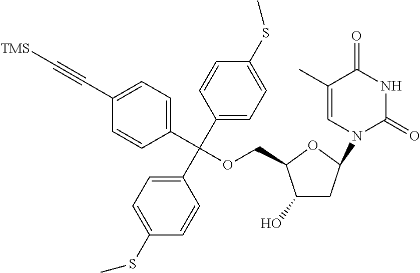 [SMILES: CSc1ccc(C(OC[C@H]2O[C@@H](n3cc(C)c(=O)[nH]c3=O)C[C@@H]2O)(c2ccc(C#C[Si](C)(C)C)cc2)c2ccc(SC)cc2)cc1]

4-Trimethylsilylethynyl-4′,4″-dimethylthiotrityl alcohol (0.30 g, 0.67 mmol) was dissolved in ethyl acetate (3.2 mL), and thionyl chloride (0.24 mL, 3.4 mmol) was added. The resulting mixture was stirred at 45° C. for 1.5 hour and then concentrated to obtain a product. Subsequently, this product was dissolved in pyridine (3.0 mL), and thymidine (73 mg, 0.30 mmol) was added. The resulting mixture was stirred at 45° C. overnight.

The stirred mixture was concentrated, and the residue was purified by silica gel column chromatography (hexane/ethyl acetate=1/1→0/1) to obtain the target compound (115 mg, 0.17 mmol) as a white solid.

¹H-NMR (DMSO-d₆): δ 11.33 (s, 1H), 7.44 (d, 1H, J=1.2 Hz), 7.43-7.36 (m, 4H), 7.29-7.20 (m, 8H), 6.20 (t, 1H, J=6.6 Hz), 5.30 (d, 1H, J=4.8 Hz), 4.28-4.21 (m, 1H), 3.90-3.86 (m, 1H), 3.22 (dd, 1H, J=5.4, 7.5 Hz), 3.13 (dd, 1H, J=3.0, 7.5 Hz), 2.45 (s, 6H), 2.28-2.19 (m, 1H), 2.16-2.08 (m, 1H), 1.54 (d, 3H, J=1.2 Hz), 0.21 (s, 9H)

¹³C-NMR (DMSO-d₆): δ 163.6, 150.4, 144.7, 139.3, 139.0, 137.5, 135.8, 131.3, 128.9, 128.8, 128.1, 125.3, 120.9, 109.6, 104.8, 94.5, 85.6, 85.2, 83.7, 70.3, 64.2, 39.4, 14.1, 11.8, −0.1

### INDUSTRIAL APPLICABILITY

The trityl-based alcohol protecting group of the present invention comprises a DMTr or like protecting group and at least one triple bond introduced into the protecting group. The trityl-based alcohol protecting group of the present invention is a novel trityl-based alcohol protecting group that is useful in synthesizing nucleic acids. The trityl-based alcohol protecting group of the compound represented by formula (1) of the present invention is excellent in controlling the ease of deprotection.

In the trityl-based alcohol protecting group of the compound represented by formula (1) of the present invention, a protecting group, such as a DMTr protecting group, changes in stability of the cation and forms a trityl-based alcohol protecting group that is easily or instantly removed, for example, under weak acidic conditions. The trityl-based alcohol protecting group of the compound represented by formula (1) of the present invention is excellent in that the triple bond introduced is detectable by Raman spectroscopy and various reactions can be tracked based on the trityl-based alcohol protecting group.

1. A compound represented by the following formula (1): 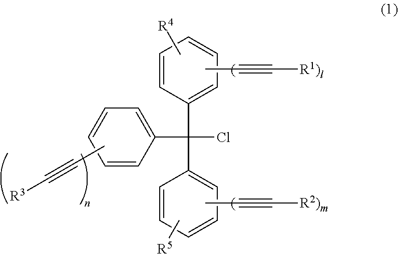
wherein
R¹, R², and R³ are same or different and each represent a hydrogen atom, an alkyl group, a trimethylsilyl group, an acetyl group, a benzoyl group, an ester group, an alkyl ester group, an aryl group, an alkyl alcohol group, a nitrile group, a group represented by 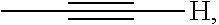
or a group represented by 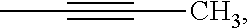
l, m, and n each represent an integer of 0, 1, or 2, provided that l, m, and n are not 0 at the same time, and
R⁴ and R⁵ are the same or different and each represent an electron-donating group.
